(19) United States
(12) Patent Application Publication
(10) Pub. No.: US 2004/0063124 A1
(43) Pub. Date: Apr. 1, 2004

(54) Methods and apparatus for preparing and assaying biological samples to determine protein concentration

(75) Inventors: Sam LaBrie, St. Peters, MO (US); Akash Mathur, St. Louis, MO (US); Kim Lawry, St. Peters, MO (US); Heather Pancio, St. Louis, MO (US); Nathan Danielson, St. Louis, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: ProteoPlex, Inc.

(21) Appl. No.: 10/636,894

(22) Filed: Aug. 6, 2003

Related U.S. Application Data
(60) Provisional application No. 60/403,624, filed on Aug. 15, 2002.

(51) Int. Cl. C12Q 1/68

(52) U.S. Cl. 435/6

(57) ABSTRACT

The present invention discloses methods and apparatus for the detection of biological molecules in various samples. In particular, a method for detecting a hapten-labeled analyte in a solution wherein substantially all other solution constituents are labeled with a hapten is disclosed. The method may be used to detect single or multiple analytes in solution. Apparatus to detect such analytes are also disclosed.

### CROSS-REFERENCES TO RELATED APPLICATIONS

This application claims priority from U.S. Provisional Application Serial No. 60/403,624 filed on Aug. 15, 2002, which is incorporated herein by reference in its entirety.

### BACKGROUND OF THE INVENTION

1. Field of the Invention

The present invention relates generally to the detection of biological molecules in various samples. In particular, the invention relates to assays that use immobilization of a single species of capture agent on a solid surface to detect its cognate binding molecule in solution. The invention relates to both systems for detecting single or multiple analytes. The methods of the invention provide a procedure for sample preparation that improves such detection. Apparatus used in such methods are also described.

2. Description of Related Art

A variety of methods exist for detecting molecules in a biological sample. Enzyme-Linked Immunosorbent Assays (ELISAs) are one of the common approaches used for diagnostic, research and screening applications. ELISAs often involve immobilizing the biological molecules of interest on a solid surface and employing an enzyme linked antibody to probe it (a direct-capture ELISA) or using an enzyme-linked antibody probe to detect a preformed complex of the molecule of interest with another antibody that is immobilized (a sandwich ELISA). Both direct-capture and sandwich ELISA methods use the enzyme linked to the antibody to produce a detectable signal when presented with its substrate. This detection method can be substituted by several others, including radio labeling, fluorescent labeling or chemiluminescent labeling of the detection antibody. Several formats for ELISA are described in U.S. Pat. No. 4,011,308 to Giaever and U.S. Pat. No. 4,016,043 to Schuurs, et al., both incorporated herein by reference in their entirety.

Biological studies sometimes call for determination of the concentration of multiple proteins of interest in a single sample (multiplex assays). Carrying out such determinations one-by-one can be time and capital intensive. Recent developments in the prior art allow simultaneous determination of multiple candidate compounds in a particular system or detection of select compounds in several systems. Several formats of such determinations, e.g., High-Throughput Screening (“HTS”) or microarrays are described in U.S. Pat. No. 6,238,869 to Kris, et. al., incorporated herein by reference in its entirety.

FIG. 1A depicts a typical sandwich ELISA or HTS protocol requiring a pair of antibodies to detect or quantitate one molecule of interest. Using this protocol, a secondary antibody is labeled for detection. The labeling reagent typically applied in this protocol is the physiological vitamin molecule biotin. The amount of biotin-labeled antibody present in a sample is detected by addition of streptavidin which binds biotin with high affinity (femtomolar Kd) and can be conjugated to detection tags such as horse radish peroxidase, alkaline phosphastase, fluorescent dyes, or other detection systems. The amount of the biotin/streptavidin complex present in a sample can then be estimated using the applicable instrumentation for that system.

Such solution-based detection systems have limited applicability in multiplex assays. Typical formats of multiplex assays involve applying several antibodies in the same well and require detection of probe bound to each antibody spot. Other multiplex applications may require pooling together of solid matrices, e.g., glass beads, from several wells and determining the amount of probe bound to each bead. In either format, solution based detection systems cannot be applied; solid phase detection systems must be used in these systems. Typically, this can be done using an anti-biotin antibody, which is itself labeled by another detectable probe that is fluorescent or chemiluminescent, among other properties (FIG. 1B).

For measurements of proteins in both ELISA and HTS formats it is required to have at least two antibodies to be available for detecting a single antigen. These two antibodies must bind to separate epitopes of the antigen so they do not compete for binding sites during the assay (matched sets). Furthermore, each antibody must have high specificity for the target antigen, i.e. it must have low to undetectable binding to similar proteins (low cross-reactivity). Multiplex assay development is even more demanding in that each antibody must not cross react with any of the target antigens of the assay. Developing matched sets of antibodies and determining which antibodies cross-react is a time consuming and inefficient process often taking months to complete. This process is often repeated even after inoculating the same species of animals using different antigens. Therefore, what is needed are methods for preparing biological samples that require fewer steps to detect or quantitate an analyte, do not require two antibodies for such detection and/or quantitation, and apparatus used in such methods.

### BRIEF SUMMARY OF THE INVENTION

This invention provides a method for detecting an analyte molecule in a solution, said analyte molecule and substantially all other solution constituents being labeled with a hapten, comprising the step of contacting the solution with a population of only one species of capture agent, said capture agent having affinity for said analyte molecule, wherein the analyte molecule is thereby detected.

This invention further provides a method for quantitating an analyte molecule in a solution, said analyte molecule and substantially all other solution constituents being labeled with a hapten, comprising the step of contacting the solution with a population of only one species of capture agent, said capture agent having affinity for said analyte molecule, and measuring the quantity of captured analyte molecules.

This invention further provides a method for quantitating at least two different analytes in solution, said analytes and substantially all other solution constituents being labeled with a hapten, comprising:

(a) contacting the solution with at least two different species of capture agents, each of said capture agents having affinity toward a different analyte; and

(b) measuring the quantity of captured analytes.

This invention provides a method for quantitating at least two different analytes in a solution comprising the steps of:

(a) attaching to a support at least two different species of capture agents, each of said capture agents having affinity toward a different analyte;

(b) labeling substantially all of the proteins in the solution with a hapten;

(c) contacting the solution with the capture agents; and

(d) measuring the quantity of captured analytes.

Further, this invention provides a method for quantitating at least two different analytes in a solution comprising the steps of:

(a) attaching to a support at least two different species of primary capture agents, each of said primary capture agents having affinity toward a different analyte;

(b) mixing the solution with an excess amount of hapten such that substantially all of the solution constituents are bound with hapten;

(c) saturating unbound hapten from step (b) such that the unbound hapten is rendered substantially inactive;

(d) contacting the solution of steps (b) and (c) with the capture agents of step (a);

(e) adding to the solution an excess amount of at least hapten binding partner exhibiting high affinity toward the hapten, said hapten binding partner being attached to a detection probe;

(f) removing unbound hapten binding partner from the biological sample; and

(g) measuring the quantity of captured analytes.

Still further, this invention provides a method for quantitating at least two different analytes in a solution using a microarray platform comprising the steps of:

(a) attaching at least two different species of capture agents, each of said capture agents having affinity toward a different analyte, to the microarray platform, said microarray platform comprising a surface gridded in a predetermined matrix of binding sites such that a predetermined number of binding sites comprise a population of a different species of capture agent exhibiting affinity toward a different analyte in the solution;

(b) labeling substantially all of the proteins in the solution with hapten;

(c) contacting the solution of step (b) with each binding site; and

(d) measuring the quantity of captured analytes.

This invention also provides a microarray platform comprising a surface gridded in a predetermined matrix of binding sites such that a predetermined number of binding sites comprise different species of capture agents exhibiting affinity toward different analyte molecules in a solution, wherein substantially all solution constituents are labeled with a detection probe, and wherein the solution is in contact with at least one binding site.

This invention further provides a kit comprising the microarray platform described immediately above. The kit may include at least one control analyte (i.e., an analyte similar to that being detected in solution which is used to compare and/or quantitate the analyte in solution), a hapten, a hapten binding partner, a detection probe, and reagents which facilitate the binding of the detection probe to the analyte in a solution. The kit may also combine these objects with a microarray platform, preferably the microarray platform described in the paragraph immediately above.

These and other objects and features of the invention will become more fully apparent when the following detailed description of the invention is read in conjunction with the accompanying figures.

### BRIEF DESCRIPTION OF THE SEVERAL VIEWS OF THE DRAWINGS

FIG. 1: Detection of biological molecules in various systems. FIG. 1A shows a typical sandwich ELISA requiring both a primary and secondary antibody to detect a biological molecule of interest. FIG. 1B shows a typical HTS format requiring both a primary and secondary antibody to detect a biological molecule of interest. FIG. 1C shown the format of the present invention requiring only one capture agent (in this Figure, an antibody) to detect, identify or quantitate a biological molecule of interest.

FIG. 2A: Effect of biotin concentration on detection of GM-CSF. pGM-CSF antibody was coated on a whole well plate. GM-CSF, IL-8 and 10% goat serum were biotinylated and detected.

FIG. 2B: Effect of biotin concentration on slope of GM-CSF standard curve. Slope of the standard curve from FIG. 2A is plotted as a function of biotin concentration. Data indicates optimal binding at 4 mM biotin, beyond which the slope declines due to increased background detection.

FIG. 3: Effect of carrier protein on biotinylation of proteins. Several different concentrations each of BSA, goat serum and fetal bovine serum (FBS) were used as carrier proteins (data for two concentrations of each is shown). The plates were coated with GM-CSF antibodies and binding of GM-CSF or IL-8 (as a control) was monitored. Data indicate that BSA allows highest signal and sensitivity followed by FBS and goat serum, which are comparable.

FIG. 4: Procedures to neutralize unbound biotin. Upon completion of the biotinylation reaction, unbound biotin was removed either using 3 kD cut-off centricon (YM-3) or neutralized by adding 50 mM Tris or 5 mM Glycine. Data indicate no substantial difference between neutralization methods.

FIG. 5: Demonstration of biotinylation of proteins and detection using the new approach in cell supernatants. Biotin labeling was carried out at several different concentrations of biotin in the pre- and post-cell media as marked in the legend. The plates were coated with GM-CSF antibodies and binding of GM-CSF or IL-8 (as a control) was monitored. Data indicate that at 4 mM biotin, proteins can be labeled and detected in the pre-and post-cell media. The sensitivity of the reaction was 62.5 pg/ml in whole well ELISAs.

FIG. 6: Application of the labeling procedure in determining concentrations of multiple analytes using microarrays. Multiple antibodies were gridded in a predetermined sequence in a microarray well. A mixture containing seven cytokines each at 1 ng/ml was biotinylated and used to determine the concentration of all analytes simultaneously. The spots were then visualized using RLS gold particles coupled with anti-biotin antibodies. A picture of the plate is shown on the left. Each column has capture antibody of one analyte in the following order (from left to right): IL-4, IL-6, IL-10, IL-2, IL-7, GM-CSF, blank, and IL-8. Each row represents different dilution of the antibody and the panel on right shows values of signal as a function of antibody concentration. The data indicate that the labeling procedure can be used to determine the concentration of multiple analytes from one biological sample.

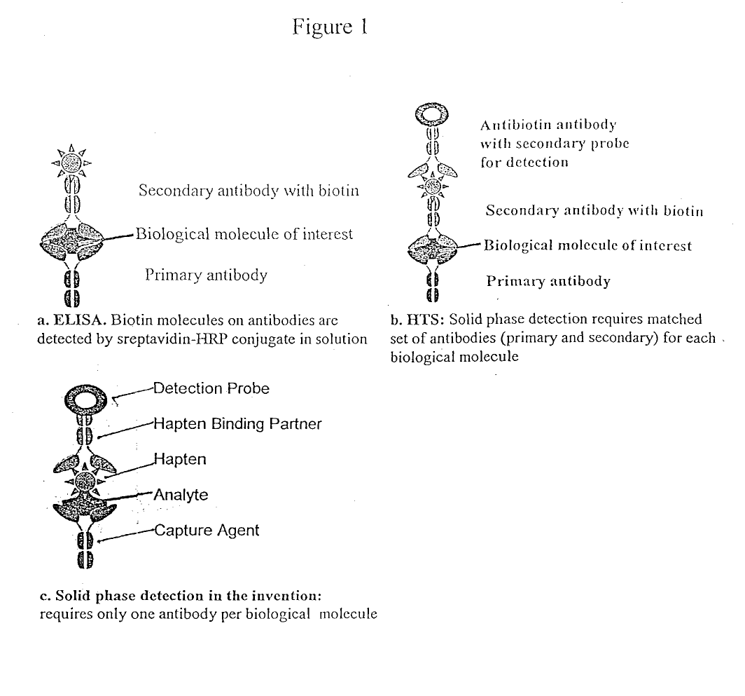

### DETAILED DESCRIPTION OF THE INVENTION

Abbreviations and Definitions

Unless indicated otherwise, the terms defined below have the following meanings:

Hapten: As used herein, the term “hapten” refers to one member of a hapten/capture agent binding pair. The hapten may be, for example, a small molecule tag added to a protein, allowing for efficient detection of that protein.

Capture Agent: As used herein, the term “capture agent” refers to the opposite member of a hapten/capture agent binding pair. The capture agent may be, for example, an antibody or antibody fragment molecule in antigen/antibody or antigen/antibody fragment binding pair. Additional examples of capture agents include, but are not limited to, an antibody, phage display, affibody, fribronectin display, antibody mimic or aptamer. Capture agents may be bound to a support according to PCT Publication No. WO 01/70641.

Analyte: As used herein, the term “analyte” or “analyte molecule” refers to a molecule, typically a macromolecule, such as a polypeptide, whose presence, amount, and/or identity are to be determined. The analyte molecule is further broadly defined to include proteins, carbohydrates, lipids, nucleic acids, small molecules and compounds, antibiotics, interleukin, enzymes, receptors, channels, pores and transcription factors or any combination thereof which is capable of being labeled by a hapten in a biological sample. Those of skill in the art will appreciate that several well-known methods of labeling the above molecules with a hapten exist which include, but is not limited to, primary amine-NHS ester linkage.

Analyte-specific assay reagent: As used herein, the term “analyte-specific assay reagent” refers to a molecule effective to bind specifically to an analyte molecule.

Detection probe: As used herein, the term “detection probe” refers to a molecule imparting a physical property which is detectable by a detector. The detection probe may be active or rendered active by an activation process, such as through an enzymatic reaction. The detection probe is bound to the hapten of the hapten/anti-hapten binding pair. A detection probe may include, for example, a fluorescent dye, chemiluminescent dye, light-scattering dye, nano-crystal, colorimetric dye, and radioactive dye or any combination thereof.

Microarray: As used herein, the term “microarray” shall mean an ordered matrix of capture agents on a support. The capture agents may be in non-overlapping areas of 0.2 to 300 μm in diameter. The matrix may be in a container to facilitate incubation with samples. Multiple containers may be combined in one device for multiple sample processing.

Ordered matrix or ordered matrices: As used herein, the “ordered matrix” or “ordered matrices” shall mean a linear or two-dimensional array of preferably discrete regions, each having a finite area, of species of capture agents formed on the surface of a support. The ordered matrix may be referred to as a “protein chip” where a microarray is printed onto a microarray platform using the printing technology of U.S. Pat. No. 6,309,891 to Shalon, et al., herein incorporated by reference in its entirety.

Microarray platform: As used herein, the term “microarray platform” refers to a support comprising at least one microarray.

Hydrophobic: A support surface is “hydrophobic” if a aqueous-medium droplet applied to the surface does not spread out substantially beyond the area size of the applied droplet. That is, the surface acts to prevent spreading of the droplet applied to the surface by hydrophobic interaction with the droplet.

Meniscus: A “meniscus” means a concave or convex surface that forms on the bottom of a liquid in a channel as a result of the surface tension of the liquid.

Biological sample: As used herein, the term “biological sample” is broadly defined to include a cell-free fluid including, but not limited to, serum, plasma, cerebrospinal fluid, lymphatic fluid, urine, sputum, semen, and synovial fluid or a protein extract including, but not limited to, a tissue extract, organ extract, and cell culture extract.

Species of capture agent: As used herein, the term “species of capture agent” shall mean an array member which is distinct from other array members on the basis of a different biopolymer sequence, and/or different concentrations of the same or distinct biopolymers, and/or different mixtures of distinct or different-concentration biopolymers. Thus, for example, an array of “species of capture agents” may comprise an array containing, as its members, (i) distinct polypeptides which may have a defined amount in each member, (ii) different, graded concentrations of given-sequence polypeptides, and/or (iii) different-composition mixtures of two or more distinct polypeptides. Each species of capture agent is said to capture a “different analyte” which includes, for example, (a) a distinct polypeptide of a solution, preferably a biological sample, and/or (b) different, graded concentrations of a distinct polypeptide of a solution, preferably a biological sample, and/or (c) a mixture of two or more distinct polypeptides of a solution, preferably a biological sample.

Biopolymer: As used herein, the term “biopolymer” is broadly defined to include polypeptides, polynucleotides, and other polymers capable of displaying specific affinity toward a corresponding binding partner through interactions including, but not limited to, static, hydrophobic, Van der Waals and ionic interactions.

Quantitating or Detecting Compounds of Interest in Solution

A procedure and apparatus has been developed to overcome the difficulties in the prior art. The procedure and apparatus for labeling samples allows for accurate determination of single and/or multiple analyte concentration in a sample for use in assays using microarrays, ELISAs and other assay methods all while using only one antibody per analyte to detect, identify and/or quantitate an analyte. This procedure is an improvement over prior art methods and apparatus because costs of production of microarrays are reduced, and steps of the assay are reduced, as well as extending applicability of assays in general to encompass analytes that have less than two validated antibodies having affinity toward the analyte.

The methods of the invention generally provide for detecting, identifying or quantitating an analyte molecule in a solution. After the analyte molecule of interest and substantially all other solution constituents are labeled with a hapten, the labeled analyte is detected, depending upon the type of hapten used, after formation of an analyte/capture agent complex. The labeling step may be accomplished in a separate volume from the capture agents, or in the same volume with the capture agents, depending on the nature of the label and capture agents. It will be appreciated by those skilled in the art that it may be determined without undue experimentation which capture agents may be present in the same volume as the labeling reaction.

The analyte/capture agent complex is formed by contacting the solution with the capture agent. Specificity of each capture agent toward a specific analyte can be determined by well-known methods in the art. It is preferable that the capture agent be highly specific toward the analyte of interest, but the identification, detection or quantitation of analytes in solution may be determined so long as the capture agent specificity is greater than its specificity toward other solution constituents. It will be appreciated by those skilled in the art that such non-specific binding, or background, may be compensated for in different manners depending on the sensitivity of the apparatus which detects the hapten or detection probe. For example, a photon detector may be able to distinguish between two hapten/analyte/capture agent complexes, thus requiring less background compensation, when the human eye cannot. Other examples of measuring the quantity of captured analytes include laser-confocal scanning, phosphorimager, geiger counter, CCD imager, spectrophotometer, and other methods known in the art capable of detecting a hapten bound to an analyte in a biological sample. Preferably, the optimal ratios of hapten, analyte and capture agent will be determined to compensate for as much background signal as possible prior to detection of the complex as described in the example below. However, detection, identification or quantitation of an analyte or analytes of interest is possible without such optimization according to the methods of the invention.

It is preferable that excess labeled analyte is used when contacting the analyte with the capture agents. In this case, the excess may be removed from contact with the capture agents by, for example, washing with a buffer.

Because this method only requires a single capture agent for the detection, identification or quantification of an analyte of interest, the number of steps required when compared to prior art assays are fewer. Thus, shorter incubation times of analyte and single capture agent versus analyte and multiple capture agents allow for more efficient processing of samples to determine whether an analyte of interest exists in a solution, and if so, in what quantity.

The methods of the invention may also be used to detect, identify and quantitate multiple analytes of interest in the same solution at the same time. The constituents of the solution are substantially all labeled with a hapten and contacted with at least two different species of capture agents. The capture agents have affinity toward different analytes of interest in the solution. The analytes are captured by the capture agents upon contacting the solution and capture agents. The number of captured analytes is measured by detecting the relative physical properties displayed by the hapten, depending on the hapten. The hapten may be required to be in contact with a hapten binding pair comprising a detection probe before it can be detected. The detection probe may be active when the hapten and hapten binding partner come into contact, such as with radiochemical detection probes, or the detection probe may become activated by the process of contacting the hapten and hapten binding partner, such as with the hapten biotin and a hapten binding partner comprising streptavidin.

The methods of quantitating multiple analytes of interest may include the steps of:

(a) attaching to a support at least two different species of capture agents, each of said capture agents having affinity toward a different analyte;

(b) labeling substantially all of the proteins in the solution with a hapten;

(c) contacting the solution with the capture agents; and

(d) measuring the quantity of captured analytes.

The labeling step, as described in the example below, is capable of labeling substantially all of the constituents of a solution containing an analyte or analytes of interest which will be identified, quantitated or detected. Such total-protein labeling was practiced in the prior art where more specific labeling of secondary antibodies was thought to be required to detect analytes of interest in a solution. However, the methods and apparatus of the present invention overcome the limitations of the prior art to provide sensitive detection of single or multiple analytes of interest in solution.

The hapten may require a hapten binding partner to become detectable. It should be noted that while introducing the additional step of contacting a hapten binding partner with a hapten decreases the overall efficiency of the detection, identification or quantitation of an analyte or analytes in solution, the present invention remains more efficient than prior art methods because it requires only one capture agent directed against each analyte of interest. A method of quantitating multiple analytes of interest where the hapten requires a hapten binding partner to render an analyte detectable may include the steps of:

(a) attaching to a support at least two different species of primary capture agents, each of said primary capture agents having affinity toward a different analyte;

(b) mixing the solution with an excess amount of hapten such that substantially all of the solution constituents are bound with hapten;

(c) saturating unbound hapten from step (b) such that the unbound hapten is rendered substantially inactive;

(d) contacting the solution of steps (b) and (c) with the capture agents of step (a);

(e) adding to the solution an excess amount of at least one hapten binding partner exhibiting high affinity toward the hapten, said hapten binding partner being attached to a detection probe;

(f) removing unbound hapten binding partner from the biological sample; and

(g) measuring the quantity of captured analytes.

Those of skill in the art will recognize that no purification step or steps is required to achieve identification, detection and/or quantitation using the methods of the present invention.

The invention may also use microarrays to quantitate an analyte or analytes in solution. Such a method may the steps of:

(a) attaching at least two different species of capture agents, each of said capture agents having affinity toward a different analyte, to the microarray platform, said microarray platform comprising a surface gridded in a predetermined matrix of binding sites such that a predetermined number of binding sites comprise a population of a different species of capture agent exhibiting affinity toward a different analyte in the solution;

(b) labeling substantially all of the proteins in the solution with hapten;

(c) contacting the solution of step (b) with each binding site; and

(d) measuring the quantity of captured analytes.

The microarray platform preferably comprises a surface gridded in a predetermined matrix of binding sites. The surface may be of any shape and may be two- or three-dimensional. Many substrates may be used including, but not limited to, glass and polymers. The predetermined number of binding sites are preferably arranged in a linear or two-dimensional format such that detection, identification or quantitation of analytes using the microarray may be easily accomplished by machine and/or the human eye.

The microarray preferably comprises different species of capture agents exhibiting affinity toward different labeled analyte molecules in a solution. A kit comprising the microarray platform described immediately above, alone or in any combination of reagents, solutions, software and instructions are also part of the invention.

The biological sample may be any tissue from a subject containing an analyte of interest. Preferably, the biological sample is either (i) a cell-free fluid including, but not limited to, a serum, plasma, cerebrospinal fluid, lymphatic fluid, urine, sputum, semen, or synovial fluid or (ii) a protein extract including, but not limited to, a tissue extract, organ extract, or cell culture extract.

The capture agents of the present invention preferably exhibit high affinity toward the analyte molecule and low affinity toward remaining solution constituents. The sensitivity of the capture agents preferably detects between 1 to 10 ng/ml analyte in a biological sample, more preferably 1 to 100 pg/ml analyte. These are referred to as “high affinity” capture agents and have dissociation constants of between 10⁻⁶ to 10⁻¹⁵ M. As such, the capture agents of the present invention may be molecules exhibiting high affinity toward an analyte or analytes of interest and include, but are not limited to, antibodies, nucleic acids, protein prints, phage displays, affibodies, fribronectin displays, antibody mimics, and aptamers. Only one capture agent is necessary to capture a distinct analyte of interest thereby allowing the detection of its presence in a sample, its identification, or quantitation of the amount of analyte present in the sample. The analyte capable of being detected by the methods and apparatus of the present invention include proteins, carbohydrates, lipids, nucleic acids, small molecules and compounds, antibiotics, or any combination thereof which are capable of being labeled by a hapten in a biological sample. Preferably, the analyte detected by the methods of the present invention may be a protein including, but not limited to, an antibody, cytokine, lymphokine or interleukin.

The analyte molecule may be detected by contacting the hapten to a hapten binding partner labeled with a detection probe such that the detection probe is rendered detectable upon such contact. In this case, the detection probe may be selected from the group consisting of a fluorescent dye, chemiluminescent dye, light-scattering dye, nano-crystal, colorimetric dye, and radioactive dye or any combination thereof. In some cases, the hapten may be labeled with a detectable probe prior to being attached to the analyte. Preferably, the hapten/hapten binding partner complex is one of (i) hapten biotin and hapten binding partner streptavidin, (ii) hapten fluorescein and hapten binding partner anti-fluorescein or (iii) hapten S peptide and hapten binding partner protein S.

Automated Apparatus for Forming Arrays

The present invention includes the formation of microarray platforms according to U.S. Pat. No. 6,110,426 to Shalon, et al., herein incorporated by reference in its entirety. Referring to the figures of this patent are helpful in fully understanding the process below. An automated apparatus according to this patent may be used for forming an array of analyte-assay regions, or binding sites, on a solid support, where each region in the array has a known amount of a selected, analyte-specific reagent.

A dispenser mounted in the device for movement toward and away from a dispensing position at which the tip of the dispenser taps a support surface is used to dispense a selected volume of reagent solution. This movement is effected by a solenoid under the control of a control unit whose operation will be described below. The solenoid is also referred to herein as dispensing means for moving the device into tapping engagement with a support, when the device is positioned at a defined array position with respect to that support.

The dispenser device is carried on an arm which is mounted on a worm screw driven (rotated) in a desired direction by a stepper motor also under control of the control unit. At its left end, a screw is carried in a sleeve for rotation about the screw axis. At its other end, the screw is mounted to the drive shaft of the stepper motor, which in turn is carried on a sleeve. The dispenser device, worm screw, the two sleeves mounting the worm screw, and the stepper motor used in moving the device in the “x” (horizontal) direction form what is referred to collectively as a displacement assembly.

The displacement assembly is constructed to produce precise, micro-range movement in the direction of the screw, i.e., along an x axis. In one mode, the assembly functions to move the dispenser in x-axis increments having a selected distance in the range 5-25 μm. In another mode, the dispenser unit may be moved in precise x axis increments of several microns or more, for positioning the dispenser at associated positions on adjacent supports, as will be described below.

The displacement assembly, in turn, is mounted for movement in the “y” (vertical) axis of the figure, for positioning the dispenser at a selected y axis position. The structure mounting the assembly includes a fixed rod mounted rigidly between a pair of frame bars, and a worm screw mounted for rotation between a pair of frame bars. The worm screw is driven (rotated) by a stepper motor which operates under the control of the control unit. The motor is mounted on a bar.

The structure just described, including the worm screw and motor, is constructed to produce precise, micro-range movement in the direction of the screw, i.e., along a y axis. As above, the structure functions in one mode to move the dispenser in y-axis increments having a selected distance in the range 5-250 μm, and in a second mode to move the dispenser in precise y-axis increments of several microns (μm) or more, for positioning the dispenser at associated positions on adjacent supports.

The displacement assembly and structure for moving this assembly in the y axis are referred to collectively as positioning means for positioning the dispensing device at a selected array position with respect to a support.

A holder in the apparatus functions to hold a plurality of supports on which the microarrays of reagent regions are to be formed by the apparatus. The holder provides a number of recessed slots which receive the supports and position them at precise selected positions with respect to the frame bars on which the dispenser moving means is mounted.

As noted above, the control unit in the device functions to actuate the two stepper motors and dispenser solenoid in a sequence designed for automated operation of the apparatus in forming a selected microarray of reagent regions on each of a plurality of supports.

The control unit is constructed, according to conventional microprocessor control principles, to provide appropriate signals to each of the solenoid and each of the stepper motors, in a given timed sequence and for appropriate signaling time. The construction of the unit, and the settings that are selected by the user to achieve a desired array pattern, will be understood from the following description of a typical apparatus operation.

Initially, one or more supports are placed in one or more slots in the holder. The dispenser is then moved to a position directly above a well containing a solution of the first reagent to be dispensed on the support(s). The dispenser solenoid is actuated now to lower the dispenser tip into this well, causing the capillary channel in the dispenser to fill. The motors are now actuated to position the dispenser at a selected array position at the first of the supports. Solenoid actuation of the dispenser is then effective to dispense a selected-volume droplet of that reagent at this location. This operation is effective to dispense a selected volume preferably between 2 pl and 2 nl of the reagent solution.

The dispenser is now moved to the corresponding position at an adjacent support and a similar volume of the solution is dispensed at this position. The process is repeated until the reagent has been dispensed at this preselected corresponding position on each of the supports.

Where it is desired to dispense a single reagent at more than two array positions on a support, the dispenser may be moved to different array positions at each support, before moving the dispenser to a new support, or solution can be dispensed at individual positions on each support, at one selected position, then the cycle repeated for each new array position.

To dispense the next reagent, the dispenser is positioned over a wash solution and the dispenser tip is dipped in and out of this solution until the reagent solution has been substantially washed from the tip. Solution can be removed from the tip, after each dipping, by vacuum, compressed air spray, sponge, or the like.

The dispenser tip is now dipped in a second reagent well, and the filled tip is moved to a second selected array position in the first support. The process of dispensing reagent at each of the corresponding second-array positions is then carried out as above. This process is repeated until an entire microarray of reagent solutions on each of the supports has been formed.

Microarray Platform

A microarray platform, or substrate, according to the invention may be formed into a two-dimensional matrix of binding sites arranged in any number of predetermined rows and columns. In one embodiment, the substrate may have an 8×12 rectangular array of cells formed on the substrate surface. Each cell in turn supports a microarray of distinct biopolymers, such as polypeptides, at known, addressable regions of the microarray.

In another embodiment, a 96-cell array typically has array dimensions between about 12 and 244 mm in width and 8 and 400 mm in length, with the cells in the array having width and length dimension of 1/12 and ⅛ the array width and length dimensions, respectively, i.e., between about 1 and 20 in width and 1 and 50 mm in length.

Preferably, the microarray platform is an ordered matrix of capture agents on a support arranged in non-overlapping areas of 0.2 to 300 μm in diameter. The matrix may be in a container to facilitate incubation with samples. Multiple containers may be combined in one device for multiple sample processing.

The substrate includes a water-impermeable backing, such as a glass slide or rigid polymer sheet. Formed on the surface of the backing is a water-permeable film. The film is formed of a porous membrane material, such as nitrocellulose membrane, or a porous web material, such as a nylon, polypropylene, or PVDF porous polymer material. The thickness of the film is preferably between about 10 and 1000 μm. The film may be applied to the backing by spraying or coating uncured material on the backing, or by applying a preformed membrane to the backing. The backing and film may be obtained as a preformed unit from commercial source, e.g., a plastic-backed nitrocellulose film available from Schleicher and Schuell Corporation.

The film-covered surface in the substrate is partitioned into a desired array of cells by water-impermeable grid lines which have infiltrated the film down to the level of the backing, and extend above the surface of the film as shown, typically a distance of 100 to 2000 μm above the film surface.

The grid lines are formed on the substrate by laying down an uncured or otherwise flowable resin or elastomer solution in an array grid, allowing the material to infiltrate the porous film down to the backing, then curing or otherwise hardening the grid lines to form the cell-array substrate.

One preferred material for the grid is a flowable silicone available from Loctite® Corporation. The barrier material can be extruded through a narrow syringe (e.g., 22 gauge) using air pressure or mechanical pressure. The syringe is moved relative to the solid support to print the barrier elements as a grid pattern. The extruded bead of silicone wicks into the pores of the solid support and cures to form a shallow waterproof barrier separating the regions of the solid support.

In alternative embodiments, the barrier element can be a wax-based material or a thermoset material such as epoxy. The barrier material can also be a UV-curing polymer which is exposed to UV light after being printed onto the solid support. The barrier material may also be applied to the solid support using printing techniques such as silk-screen printing. The barrier material may also be a heat-seal stamping of the porous solid support which seals its pores and forms a water-impervious barrier element. The barrier material may also be a shallow grid which is laminated or otherwise adhered to the solid support.

In addition to plastic-backed nitrocellulose, the solid support can be virtually any porous membrane with or without a non-porous backing. Such membranes are readily available from numerous vendors and are made from nylon, PVDF, polysulfone and the like. In an alternative embodiment, the barrier element may also be used to adhere the porous membrane to a non-porous backing in addition to functioning as a barrier to prevent cross contamination of the assay reagents.

In an alternative embodiment, the solid support can be of a non-porous material. The barrier can be printed either before or after the microarray of biomolecules is printed on the solid support.

As can be appreciated, the cells formed by the grid lines and the underlying backing are water-impermeable, having side barriers projecting above the porous film in the cells. Thus, defined-volume samples can be placed in each well without risk of cross-contamination with sample material in adjacent cells.

As noted above, each well contains a microarray of distinct biopolymers. In one general embodiment, the microarrays in the well are identical arrays of distinct biopolymers, e.g., different sequence polypeptides. Such arrays can be formed by depositing a first selected polypeptide at the same selected microarray position in each of the cells, then depositing a second polypeptide at a different microarray position in each well, and so on until a complete, identical microarray is formed in each cell.

In a preferred embodiment, each microarray contains about 10³ distinct polypeptide biopolymers per surface area of less than about 1 cm². Also in a preferred embodiment, the biopolymers in each microarray region are present in a defined amount between about 0.1 femtomoles and 100 nanomoles.

Each assay may be conducted in an “open-face” format where no further sealing step is required, since the hybridization solution will be kept properly hydrated by the water vapor in the humid chamber. At the conclusion of the incubation step, the entire solid support containing the numerous microarrays is rinsed quickly enough to dilute the assay reagents so that no significant cross contamination occurs. The entire solid support is then reacted with detection reagents if needed and analyzed using standard colorimetric, radioactive or fluorescent detection means. All processing and detection steps are performed simultaneously to all of the microarrays on the solid support ensuring uniform assay conditions for all of the microarrays on the solid support.

### EXAMPLES

The following experimental examples are offered by way of illustration and not by way of limitation.

### Example 1

Total Protein Labeling with Biotin

Biotin was used as the detection reagent to identify the presence of one or more analytes of interest using the following steps:

(a) providing an antibody on a solid support;

(b) labeling a solution which contains an analyte of interest with biotin, such that substantially all protein molecules are labeled with the detection probe;

(c) saturating and rendering any unbound detection probe unreactive;

(d) detecting the presence of an antibody/biotinylated protein complex, if present, by using antibodies labeled with a secondary biotin detection reagent.

This method was applied to assays detecting single and multiple analytes simultaneously.

Providing the Antibody on a Support

Providing the antibody on a support was performed with by the apparatus and methods disclosed in U.S. Pat. No. 6,110,426 to Shalon, et al., as described above.

Labeling the Solution with Biotin

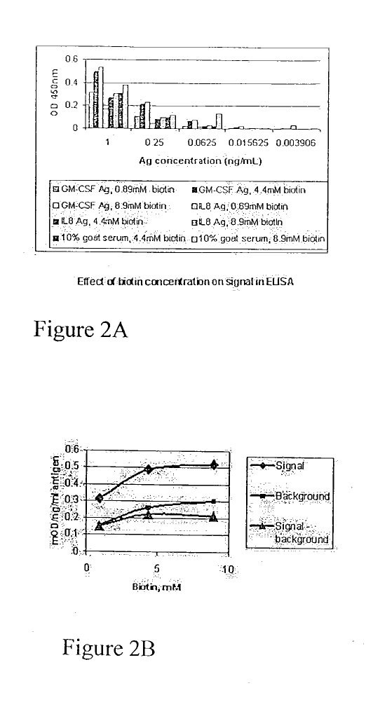

Two different sources of biotin were tested: EZ-Link™ from Pierce (catalog # 21420) and Biotin labeling kit from Roche (catalog # 1418165). Both kits employ biotin-N-hydroxyl-succinamide ester (NHS ester) linkage that labels primary amino groups with biotin, including amino-terminus and Lysine side chains. Identical results were obtained with both kits (only Roche Biotin labeling kit data is shown; FIGS. 2A and 2B). The following variables were standardized:

Concentration of Biotin Employed

For these experiments the antigen (GM-CSF or IL-8) was biotinylated using 10% fetal calf serum (FCS) as carrier protein. After a two-hour incubation with different concentrations of biotin, the proteins were used as standards in whole well ELISAs and amount of biotin-antigen bound to antibody was determined using Streptavidin-HRP. The data is show in FIG. 2. The data indicates that the slope of the standard curve is saturation beyond 4.4 mM biotin and the background slightly increased, thus resulting in an optimal labeling at 4.4 mM biotin (FIG. 2B).

Carrier Proteins Employed

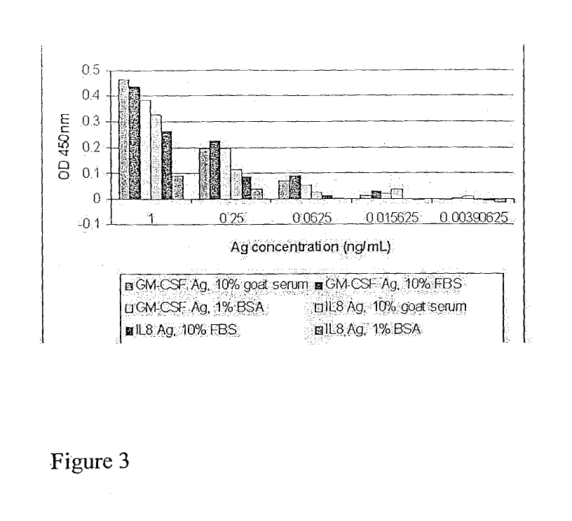

Three different carrier proteins were employed: 1% BSA, 10% FCS and 10% goat Serum (GS). 4.4 mM biotin was found to be optimal for all the three carrier proteins (FIG. 3).

Saturating and Rendering Any Unbound Detection Probe Unreactive

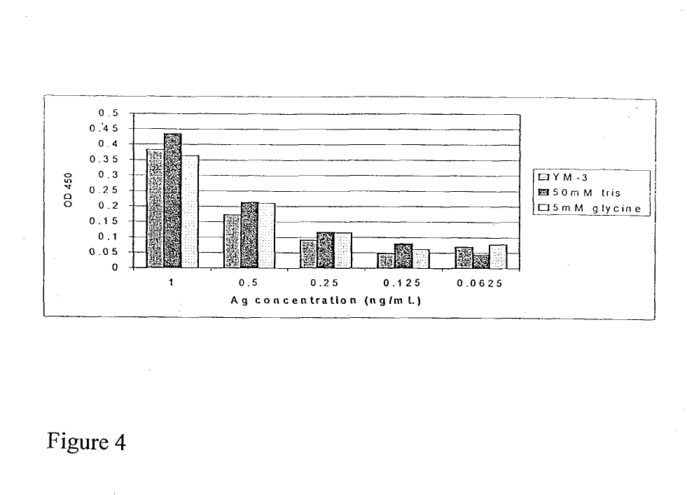

The unbound biotin in the sample can react with the gridded antibody and blocking proteins. Moreover, the recommended procedure for removing unbound biotin is a gel filtration column. While this method provides very good separation, it is time consuming. Secondly, for use with multiple analytes, the mixture of labeled proteins will typically elute as different peaks on the gel filtration column and will result in loss or dilution of sample. Accordingly, two alternative approaches were tested to remove unreacted biotin: (1) removing on a 3 kD cut-off membrane filter, and (2) neutralizing the biotin with primary amines of smaller molecular weight chemicals such as Tris or glycine. The data, shown in FIG. 4, indicate that 50 mM Tris neutralizes unbound biotin as well as physical separation using the 3 kD cutoff membrane filter. In addition, this approach uses less time and allows for better quantitation and can be used for a large number of samples. The data also indicate that neutralization of biotin with Tris gave comparable results to physical purification removing the free label.

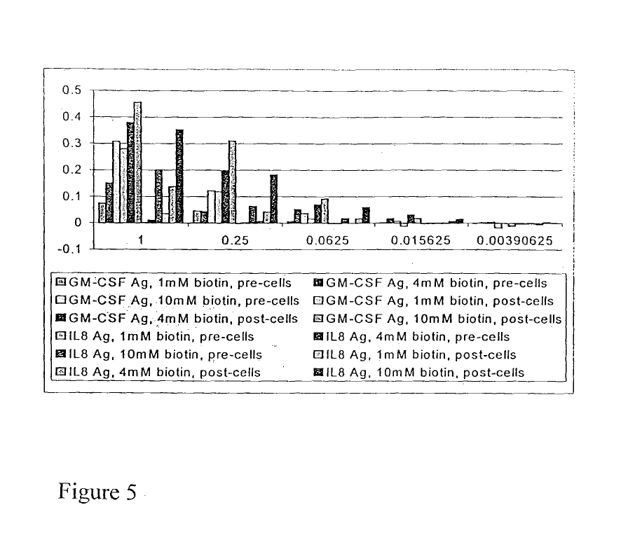

Next, in order to simulate the reagent conditions of the samples, various cytokines were added to cell culture supernatants used in ELISAs. These data are presented in FIG. 5. The data indicates that the procedure can be used to biotinylate the samples in cell culture supernatants.

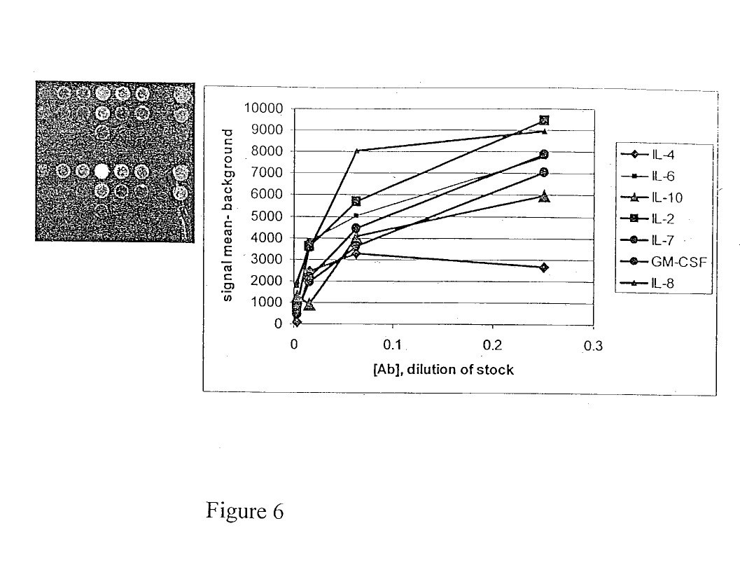

Optimal conditions were obtained using the above experiments in microarrays. The protocols established above were equally applicable to the microarray system and ELISA. FIG. 6 shows the application of the labeling procedure in determining concentrations of multiple analytes using microarrays. Multiple antibodies were gridded in a predetermined sequence in a microarray well. A mixture containing seven cytokines each at 1 ng/ml was biotinylated and used to determine the concentration of all analytes simultaneously. The spots were then visualized using RLS gold particles coupled with anti-biotin antibodies. A picture of the plate is shown on the left. Each column has capture antibody of one analyte in the following order (from left to right): IL-4, IL-6, IL-10, IL-2, IL-7, GM-CSF, blank, and IL-8. Each row represents different dilutions of the antibody and the panel on right shows values of signal as a function of antibody concentration. The data indicate that the labeling procedure of the present invention can be used to determine the concentration of multiple analytes from one biological sample (FIG. 6).

Other Embodiments

The invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

References Cited

All references cited above are incorporated herein by reference in their entirety and for all purposes to the same extent as if each individual publication, patent or patent application was specifically and individually indicated to be incorporated by reference in its entirety for all purposes. Citation of a reference herein shall not be construed as an admission that such is prior art to the present invention.

What is claimed is:

1. A method for detecting an analyte molecule in a solution, said analyte molecule and substantially all other solution constituents being labeled with a hapten, comprising the step of contacting the solution with a population of only one species of capture agent, said capture agent having affinity for said analyte molecule, wherein the analyte molecule is thereby detected.

2. The method of claim 1, wherein the solution is a biological sample.

3. The method of claim 2, wherein the biological sample is a cell-free fluid selected from the group consisting of a serum, plasma, cerebrospinal fluid, lymphatic fluid, urine, sputum, semen, and synovial fluid.

4. The method of claim 2, wherein the biological sample is a protein extract selected from the group consisting of a tissue extract, organ extract, and cell culture extract.

5. The method of claim 1, wherein the capture agent exhibits high affinity toward the analyte molecule and low affinity toward remaining solution constituents.

6. The method of claim 1, wherein the capture agent is selected from the group consisting of an antibody, nucleic acid, protein print, phage display, affibody, fribronectin display, antibody mimic, and aptamer.

7. The method of claim 1, wherein the analyte molecule is selected from the group consisting of an antibody, cytokine, lymphokine, interleukin, enzymes, receptors, channels, pores and transcription factors.

8. The method of claim 1, wherein the analyte molecule is detected by contacting the hapten to a hapten binding partner labeled with a detection probe such that the detection probe is rendered detectable upon such contact, and wherein the detection probe is detected.

9. The method of claim 8, wherein the detection probe is selected from the group consisting of a fluorescent dye, chemiluminescent dye, light-scattering dye, nano-crystal, colorimetric dye, and radioactive dye or any combination thereof.

10. The method of claim 8, wherein the hapten is biotin and the hapten binding partner is streptavidin, the hapten is fluorescein and the hapten binding partner is anti-fluorescein or the hapten is S peptide and the hapten binding partner is protein S.

11. A method for quantitating an analyte molecule in a solution, said analyte molecule and substantially all other solution constituents being labeled with a hapten, comprising the step of contacting the solution with a population of only one species of capture agent, said capture agent having affinity for said analyte molecule, and measuring the quantity of captured analyte molecules.

12. The method of claim 11, wherein the solution is a biological sample.

13. The method of claim 12, wherein the biological sample is a cell-free fluid selected from the group consisting of a serum, plasma, cerebrospinal fluid, lymphatic fluid, urine, sputum, semen, and synovial fluid.

14. The method of claim 12, wherein the biological sample is a protein extract selected from the group consisting of a tissue extract, organ extract, and cell culture extract.

15. The method of claim 11, wherein the capture agent exhibits high affinity toward the analyte molecule and low affinity toward remaining solution constituents.

16. The method of claim 11, wherein the capture agent is selected from the group consisting of an antibody, nucleic acid, protein print, phage display, affibody, fribronectin display, antibody mimic, and aptamer.

17. The method of claim 11, wherein the analyte molecule is selected from the group consisting of an antibody, cytokine, lymphokine, interleukin, enzymes, receptors, channels, pores and transcription factors.

18. The method of claim 11, wherein the analyte molecule is detected by contacting the hapten to a hapten binding partner labeled with a detection probe such that the detection probe is rendered detectable upon such contact, and wherein the detection probe is detected.

19. The method of claim 18, wherein the detection probe is selected from the group consisting of a fluorescent dye, chemiluminescent dye, light-scattering dye, nano-crystal, colorimetric dye, and radioactive dye or any combination thereof.

20. The method of claim 18, wherein the hapten is biotin and the hapten binding partner is streptavidin, the hapten is fluorescein and the hapten binding partner is anti-fluorescein or the hapten is S peptide and the hapten binding partner is protein S.

21. A method for quantitating at least two different analytes in solution, said analytes and substantially all other solution constituents being labeled with a hapten, comprising:
(a) contacting the solution with at least two different species of capture agents, each of said capture agents having affinity toward a different analyte; and
(b) measuring the quantity of captured analytes.

22. The method of claim 21, wherein the solution is a biological sample.

23. The method of claim 22, wherein the biological sample is a cell-free fluid selected from the group consisting of a serum, plasma, cerebrospinal fluid, lymphatic fluid, urine, sputum, semen, and synovial fluid.

24. The method of claim 22, wherein the biological sample is a protein extract selected from the group consisting of a tissue extract, organ extract, and cell culture extract.

25. The method of claim 21, wherein each of said species of capture agents exhibits high affinity toward a different analyte in the solution and low affinity toward remaining solution constituents.

26. The method of claim 21, wherein the capture agent is selected from the group consisting of antibody, nucleic acid, protein print, phage display, affibody, fribronectin display, antibody mimic, and aptamer.

27. The method of claim 21, wherein the analyte is selected from the group consisting of an antibody, cytokine, lymphokine, interleukin, enzymes, receptors, channels, pores and transcription factors.

28. The method of claim 21, wherein the analyte is detected by contacting the hapten to a hapten binding partner labeled with a detection probe such that the detection probe is rendered detectable upon such contact, and wherein the detection probe is detected.

29. The method of claim 28, wherein the detection probe is selected from the group consisting of a fluorescent dye, chemiluminescent dye, light-scattering dye, nano-crystal, colorimetric dye, and radioactive dye or any combination thereof.

30. The method of claim 28, wherein the hapten is biotin and the hapten binding partner is streptavidin, the hapten is fluorescein and the hapten binding partner is anti-fluorescein, or the hapten is S peptide and the hapten binding partner is protein S.

31. A method for quantitating at least two different analytes in a solution comprising the steps of:
(a) attaching to a support at least two different species of capture agents, each of said capture agents having affinity toward a different analyte;
(b) labeling substantially all of the analytes in the solution with a hapten;
(c) contacting the solution with the capture agents; and
(d) measuring the quantity of captured analytes.

32. The method of claim 31, wherein the solution is a biological sample.

33. The method of claim 32, wherein the biological sample is a cell-free fluid selected from the group consisting of a serum, plasma, cerebrospinal fluid, lymphatic fluid, urine, sputum, semen, and synovial fluid.

34. The method of claim 32, wherein the biological sample is a protein extract selected from the group consisting of a tissue extract, organ extract, and cell culture extract.

35. The method of claim 31, wherein each of said species of capture agents exhibits high affinity toward a different analyte in the solution and low affinity toward remaining solution constituents.

36. The method of claim 31, wherein the capture agent is selected from the group consisting of antibody, nucleic acid, protein print, phage display, affibody, fribronectin display, antibody mimic, and aptamer.

37. The method of claim 31, wherein the analyte is selected from the group consisting of an antibody, cytokine, lymphokine, interleukin, enzymes, receptors, channels, pores and transcription factors.

38. The method of claim 31, wherein the analyte is detected by contacting the hapten to a hapten binding partner labeled with a detection probe such that the detection probe is rendered detectable upon such contact, and wherein the detection probe is detected.

39. The method of claim 38, wherein the detection probe is selected from the group consisting of a fluorescent dye, chemiluminescent dye, light-scattering dye, nano-crystal, colorimetric dye, and radioactive dye or any combination thereof.

40. The method of claim 38, wherein the hapten is biotin and the hapten binding partner is streptavidin, the hapten is fluorescein and the hapten binding partner is anti-fluorescein, or the hapten is S peptide and the hapten binding partner is protein S.

41. A method for quantitating at least two different analytes in a solution comprising the steps of:
(a) attaching to a support at least two different species of capture agents, each of said capture agents having affinity toward a different analyte;
(b) mixing the solution with an excess amount of hapten such that substantially all of the solution constituents are bound with hapten;
(c) saturating unbound hapten from step (b) such that the unbound hapten is rendered substantially inactive;
(d) contacting the solution of steps (b) and (c) with the capture agents of step (a);
(e) adding to the solution an excess amount of at least one hapten binding partner exhibiting high affinity toward the hapten, said hapten binding partner being attached to a detection probe;
(f) removing unbound hapten binding partner from the biological sample; and
(g) measuring the quantity of captured analytes.

42. The method of claim 41, wherein the solution is a biological sample.

43. The method of claim 42, wherein the biological sample is a cell-free fluid selected from the group consisting of a serum, plasma, cerebrospinal fluid, lymphatic fluid, urine, sputum, semen, and synovial fluid.

44. The method of claim 42, wherein the biological sample is a protein extract selected from the group consisting of a tissue extract, organ extract, and cell culture extract.

45. The method of claim 41, wherein each of said species of capture agents exhibits high affinity toward a different analyte in the solution and low affinity toward remaining solution constituents.

46. The method of claim 41, wherein the capture agent is selected from the group consisting of antibody, nucleic acid, protein print, phage display, affibody, fribronectin display, antibody mimic, and aptamer.

47. The method of claim 41, wherein the analyte is selected from the group consisting of an antibody, cytokine, lymphokine, interleukin, enzymes, receptors, channels, pores and transcription factors.

48. The method of claim 41, wherein detection probe is activated by contacting the attached hapten binding partner and hapten such that the detection probe is rendered detectable upon such contact.

49. The method of claim 48, wherein the detection probe is selected from the group consisting of a fluorescent dye, chemiluminescent dye, light-scattering dye, nano-crystal, colorimetric dye, and radioactive dye or any combination thereof.

50. The method of claim 48, wherein the hapten is biotin and the hapten binding partner is streptavidin, the hapten is fluorescein and the hapten binding partner is anti-fluorescein, or the hapten is S peptide and the hapten binding partner is protein S.

51. A method for quantitating at least two different analytes in a solution using a microarray platform comprising the steps of:
(a) attaching at least two different species of capture agents to the microarray platform, said microarray platform comprising a multi-well tray, such that each well of the multi-well tray contains an ordered matrix of species of capture agents;
(b) labeling substantially all of the analytes in the solution with hapten;
(c) contacting the solution of step (b) with each binding site; and
(d) measuring the quantity of captured analytes.

52. The method of claim 51, wherein the solution is a biological sample.

53. The method of claim 52, wherein the biological sample is a cell-free fluid selected from the group consisting of a serum, plasma, cerebrospinal fluid, lymphatic fluid, urine, sputum, semen, and synovial fluid.

54. The method of claim 52, wherein the biological sample is a protein extract selected from the group consisting of a tissue extract, organ extract, and cell culture extract.

55. The method of claim 51, wherein each of said species of capture agents exhibits high affinity toward a different analyte in the solution and low affinity toward remaining solution constituents.

56. The method of claim 51, wherein the capture agent is selected from the group consisting of antibody, nucleic acid, protein print, phage display, affibody, fribronectin display, antibody mimic, and aptamer.

57. The method of claim 51, wherein the analyte is selected from the group consisting of an antibody, cytokine, lymphokine, interleukin, enzymes, receptors, channels, pores and transcription factors.

58. The method of claim 51, wherein the analyte is detected by contacting the hapten to a hapten binding partner labeled with a detection probe such that the detection probe is rendered detectable upon such contact, and wherein the detection probe is detected.

59. The method of claim 58, wherein the detection probe is selected from the group consisting of a fluorescent dye, chemiluminescent dye, light-scattering dye, nano-crystal, colorimetric dye, and radioactive dye or any combination thereof.

60. The method of claim 58, wherein the hapten is biotin and the hapten binding partner is streptavidin, the hapten is fluorescein and the hapten binding partner is anti-fluorescein, or the hapten is S peptide and the hapten binding partner is protein S.

61. A microarray platform comprising different species of capture agents exhibiting affinity toward different analyte molecules in a solution, wherein substantially all solution constituents are labeled with a hapten, and wherein the solution is in contact with at least one capture agent.

62. The microarray platform of claim 61, wherein the surface comprises a multi-well tray such that each well of the multi-well tray contains an ordered matrix of species of capture agents.

63. The microarray platform of claim 61, wherein the binding sites are arranged in identical ordered matrices.

64. The microarray platform of claim 62, wherein each of said species of capture agents is gridded in a series of graded concentrations.

65. The microarray platform of claim 62, wherein each of said species of capture agents is gridded as mixtures of two or more distinct capture agents.

66. The microarray platform of claim 61, wherein the hapten is in contact with a hapten binding partner labeled with a detection probe such that the detection probe is rendered detectable upon such contact.

67. The method of claim 66, wherein the detection probe is selected from the group consisting of a fluorescent dye, chemiluminescent dye, light-scattering dye, nano-crystal, colorimetric dye, and radioactive dye or any combination thereof.

68. The method of claim 66, wherein the hapten is biotin and the hapten binding partner is streptavidin, the hapten is fluorescein and the hapten binding partner is anti-fluorescein, or the hapten is S peptide and the hapten binding partner is protein S.

69. The microarray platform of claim 61, wherein the capture agent exhibits high affinity toward the detection probe.

70. The microarray platform of claim 61, wherein the capture agent is selected from the group consisting of antibody, nucleic acid, protein print, phage display, affibody, fribronectin display, antibody mimic, and aptamer.

71. The microarray platform of claim 61, wherein the analyte molecule is selected from the group consisting of an antibody, cytokine, lymphokine, interleukin, enzymes, receptors, channels, pores and transcription factors.

72. A kit comprising the microarray platform of claim 61.

73. A kit for detecting the concentration of an analyte in a solution comprising at least one control analyte, a hapten, a hapten binding partner, a detection probe, and reagents, wherein said reagents facilitate the binding of the detection probe to the analyte in a solution.

74. The kit of claim 73 further comprising a microarray platform.

75. The kit of claim 74, wherein the platform is the microarray platform of claim 61.
